(19) United States
(12) Patent Application Publication
(10) Pub. No.: US 2026/0209235 A1
(43) Pub. Date: Jul. 23, 2026

(54) Purification of triazabicyclodecene (TBA)

(71) Applicant: Aalto University Foundation sr, Aalto (FI)

(72) Inventors: Ville Alopaeus, Aalto (FI); Gao Ming Jerald Foo, Aalto (FI); Inge Schlapp-Hackl, Aalto (FI); Herbert Sixta, Aalto (FI); Petri Uusi-Kyyny, Aalto (FI)

(21) Appl. No.: 19/139,379

(22) PCT Filed: Dec. 15, 2023
(86) PCT No.: PCT/FI2023/050702

§ 371 Date: Jun. 16, 2025

(30) Foreign Application Priority Data
Dec. 16, 2022 (FI) 20226116

(51) Int. Cl. C07D 487/04; B01D 3/10; B01D 11/02

(52) U.S. Cl. CPC C07D 487/04; B01D 3/10; B01D 11/02

(57) ABSTRACT

According to an example aspect of the present invention, there is provided a method for separating mTBD from a sample comprising mTBD and TBD, wherein the method comprises the steps of filtering the sample, and subsequently distilling the sample.

### FIELD

The present invention relates to purification of guanidine bases. More specifically, the present invention relates to purification of 7-Methyl-1,5,7-Triazabicyclo[4.4.0]dec-5-ene (mTBD).

### BACKGROUND

Ioncell technology can be used for the transformation of cellulosic fibers derived for example from used textiles, pulp, or old newspapers into fibers that can be used for example for the production of new knitted and woven garments. The Ioncell process utilizes an ionic liquid to dissolve cellulose, which can then be transformed into fibers using dry-jet wet spinning technology (Elsayed et al.).

Ionic liquid is a salt in a liquid state. One suitable ionic liquid for the Ioncell process is mTBD, which is a bicyclic strong guanidine base. It forms an ionic liquid when brought in contact with certain acids.

TBD is an impurity in mTBD preparations. TBD has the potential to form solid particles in the Ioncell process and also increases the melting point of the ionic liquid produced thereof. For these reasons, the TBD content of mTBD preparations should be reduced to as low level as possible before producing the ionic liquid. In addition, hydrolysis products of m TBD should be removed from mTBD preparations used for the Ioncell process. The removal of the hydrolysis products is crucial for the subsequent use of the preparation in cellulose dissolution.

The cost of the TBD-containing mTBD superbase is significantly lower than that of pure mTBD preparations. However, to our knowledge there is no known method for easy and cost-efficient purification of mTBD from preparations containing TBD.

### SUMMARY OF THE INVENTION

The invention is defined by the features of the independent claims. Some specific embodiments are defined in the dependent claims.

According to a first aspect of the present invention, there is provided a method for separating mTBD from a sample comprising mTBD and TBD, wherein the method comprises the steps of filtering the sample, and subsequently distilling the sample.

The present invention is based on the novel finding that mTBD and TBD can be separated based on their difference in vapour pressure. This is surprising since the two compounds are structurally very similar and thus person skilled in the art would not expect that distillation is a viable strategy for separating these compounds. In addition, the molar mass of TBD is lower than that of mTBD and still surprisingly, TBD has much lower vapour pressure. This makes it possible to obtain very pure mTBD by distillation.

### BRIEF DESCRIPTION OF THE DRAWINGS

FIG. 1 presents composition of the samples taken during distillation. y-axis present mass-% of components (%) calculated from the total mass of the sample, x-axis presents cumulative mass of distillate (g), black line presents mTBD, grey line present H-mTBD-2 and dotted line H-mTBD-1.

FIG. 2 illustrates the solid liquid equilibria of TBD and mTBD measured with differential scanning calorimetry. y-axis presents temperature (K) and x-axis TBD equilibria. The line in the Figure presents ideal equilibria, circle presents pure mTBD, x presents differential scanning calorimeter measurement, and triangle room temperature solubility of TBD in mTBD.

FIG. 3 presents a schematic set up of the batch column used in the example. 1. Heater, 2. Round bottom flask, 3. Vigreaux column, 4. distillate reflux controller, 5. Condenser, 6. Additional distillate condenser, 7. Round bottom flasks for distillate fraction collection, 8. vacuum line, 9. Liquid nitrogen trap, 10 Vacuum control valve, T temperature probes, P pressure transducer.

FIG. 4 present temperature profile of the distillation column. y-axis presents temperature (° C.), x-axis present time (min), line present reboiler temperature and dotted line presents temperature at the top of the column.

FIG. 5 presents pressure of the distillation column. y-axis presents pressure (mbar), x-axis presents time (min).

FIG. 6 illustrates vapour pressure of mTBD and TBD. y-axis present vapour pressure (Pa), x-axis temperature (° C.), circle presents mTBD vapour pressure measured by Baird et al (2019), triangles present measured mTBD vapour pressure, box present measured TBD vapour pressure and line present TBD correlation.

### EMBODIMENTS

In the present context, the abbreviation “TBD” refers to 1,5,7-Triazabicyclo[4.4.0]dec-5-ene for which the CAS number is 84030-20-6. The abbreviations “m-TBD” and “mTBD” refer to 7-Methyl-1,5,7-Triazabicyclo[4.4.0]dec-5-ene for which the CAS number is 5807-14-7. The abbreviation H-mTBD-1 refers to 1-[3-methylammonio) propyl]-1,3-diazinan-2-one. The abbreviation H-mTBD-2 refers to 1-[3-ammonio) propyl]-3-methyl-1,3-diazinan-2-one. H-mTBD-1 and H-mTBD-2 are hydrolysis products of mTBD.

In the present context, the term “ionic liquid” refers to salts showing liquid properties at room temperature. These substances are also known with the names liquid electrolytes, ionic melts, ionic fluids, fused salts, liquid salts, or ionic glasses. While ordinary liquids are predominantly made of electrically neutral molecules, ionic liquids are largely made of ions.

Unless otherwise stated herein or clear from the context, any percentages referred to herein are expressed as percent by weight based on a total weight of the respective composition.

Unless otherwise stated, properties that have been experimentally measured or determined herein have been measured or determined at room temperature. Unless otherwise indicated, room temperature is 25° C. Unless otherwise stated, properties that have been experimentally measured or determined herein have been measured or determined at atmospheric pressure.

As used herein, the term “about” refers to the actual given value, and also to an approximation to such given value that would reasonably be inferred to one of ordinary skill in the art, including approximations due to the experimental and/or measurement conditions for such given value. Introductory paragraph—explain the crux of the invention here in one paragraph to provide an executive summary of what the application in general is about.

The present disclosure relates to a method for separating mTBD from a sample comprising mTBD and TBD. The method comprises the steps of filtering the sample, and subsequently distilling the sample.

The filtration can be performed by applying the sample to a filtering medium and collecting the fluid passing through. The filtering medium can be any medium suitable for separating solid material from liquid, such as filter paper or filtering membrane. In one exemplary set-up, a filter paper is placed to a glass funnel and pre-wetted, the sample is applied onto the pre-wetted filter paper and the fluid that passes through the filter paper and contains mTBD is collected. The filter cake forming on top of the filter paper comprises TBD. The filter cake may in some embodiments be collected as TBD enriched fraction. The filter medium may be washed after passing the sample through to recover any sample remaining in the filtering medium. Wash solution is applied onto the filtering medium and fluid passing through is collected as wash fraction and may be combined with the sample that was collected after filtering. Filtering maybe performed for example as gravity filtration or as vacuum filtration.

In some preferred embodiments, TBD content of the sample is 5 mol-% or less, such as 3 mol-% or less, 2 mol-% or less, calculated from the combined moles of mTBD and TBD in the sample after filtration. In some examples, TBD concentration may be further reduced by lowering the storage temperature of the sample according to solid liquid equilibria behaviour of TBD (FIG. 2). Preferably, the storage temperature ranges from 16° C. to 25° C., such as from 16° C. to 20° C. In some embodiments the filtering step may be performed at a temperature ranging from 10 to 30° C. Preferably, the temperature may be between 12-25° C., most preferably between 16-20° C. At higher temperatures more TBD is dissolved in the mTBD solution (FIG. 2). Therefore, lower temperatures are beneficial for the separation of TBD from mTBD solution. At temperatures below 16° C., the sample containing mTBD may be brought to subcooled state.

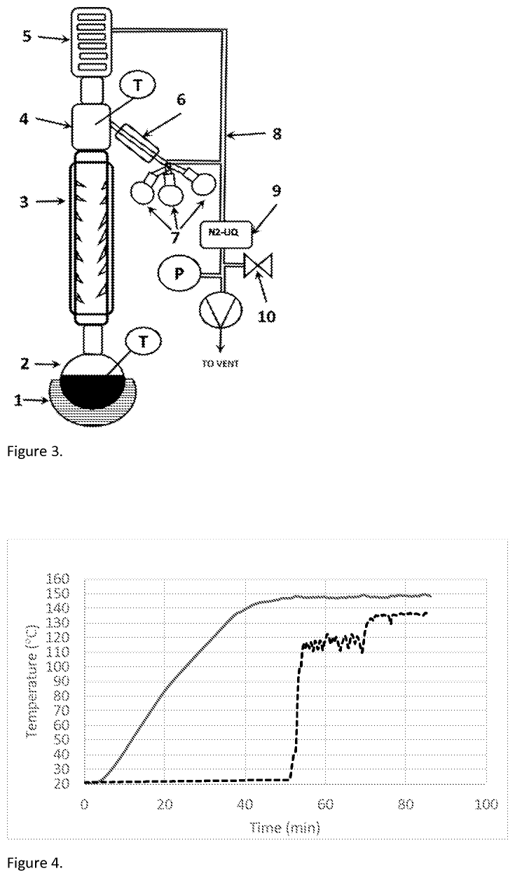

The set-up for the distillation device can be simple, and include a flask for holding the material to be distilled, a heater and a reflux column. However, more complex devices are typically used, and are known in the art. One exemplary set-up comprises a reboiler flask, a column and column packing, and a condenser with a reflux controller. Another exemplary set-up is defined in FIG. 3, and includes 1. Heater, 2. Round bottom flask, 3. Vigreaux column, 4. distillate reflux controller, 5. Condenser, 6. Additional distillate condenser, 7. Round bottom flasks for distillate fraction collection, 8. vacuum line, 9. Liquid nitrogen trap, 10 Vacuum control valve, T temperature probes, P pressure transducer. Thus, the device can be a batch distillation column, a continuous distillation column, a short path distillation unit, thin film evaporator or any combination thereof. The thin film evaporator may be selected from a group comprising batch evaporator, natural circulation evaporator, forced circulation evaporator, horizontal tube evaporator, long tube vertical evaporator, short tube vertical evaporator, falling film evaporator, rising film evaporator, rising/falling film evaporator, agitated film evaporator and gasketed plate evaporator.

The distillation device is typically operated by introducing the feed into the reboiler flask, and connecting the flask to the column. After heating has begun, distillate fractions are taken out from the top of the column into the round bottom flask. In a batch distillation procedure, as used herein, the first 1-2 fractions typically contain water, and are therefore preferably discarded. The following fractions, e.g. fractions 3-7, however, consist of mainly mTBD, and are thus collected as product fractions.

In some embodiments, TBD present in the sample may be precipitated and the precipitate removed prior to distillation. Precipitation may be achieved for example by reducing the temperature of the solution to temperatures below room temperature, for example in the range of 12-25° C. or 16-20° C. Preferably, the precipitate may be removed by filtering or decanting. In some embodiments, the temperature during the removal of the precipitate is 10-30° C., preferably 12-25° C., most preferably 16-20° C. Precipitation of TBD will reduce the TBD concentration in the sample and ultimately yield in more efficient purification of the sample.

In some embodiments, the pressure during distillation may be below 101.325 kPa. Preferably, the pressure during distillation may be 1-10 kPa, preferably 1-5 kPa, most preferably 2-2.5 kPa. At reduced pressure, water cannot condense, and it is transferred to a trap. In addition, hydrolysis products of mTBD, namely H-mTBD-1 and H-mTBD-2, are also directed to the trap under reduced pressure. Therefore, lowering the pressure during distillation will improve the purity of the mTBD preparation.

In other embodiments, the sample is dried before distillation, among others to reduce reactions with water, forming unwanted byproducts. In the presence of water, the mTBD hydrolyses to H1-mTBD and H2-mTBD, decreasing its dissolving power for cellulose. Further, the drying step reduces water content of the ionic liquid. Excess water in the ionic liquid decreases the dissolving power of the ionic liquid for cellulose. Thus, the drying step may increase the purity of mTBD. The drying is preferably carried out by stripping the sample with an inert gas such as nitrogen gas, by heating, by evaporating, by freeze-drying or by combinations thereof.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that the terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as if each member of the list were individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### EXPERIMENTAL SECTION

The sample contains a mixture of 1,5,7-Triazabicyclo[4.4.0]dec-5-ene (TBD) and 7-Methyl-1,5,7-Triazabicyclo[4.4.0]dec-5-ene (mTBD). The column was a single section set-up with Montz packing Type A3-1000 (7×80 mm) internals. The dimension of the column was a DN25 diameter silver coated with vacuum isolation, sight stripes and outer expansion bellows. The filling height was 560 mm and total height ca. 690 mm. The absolute pressure on the column side was set to Pabs, distcol=21 mbar. The target pressure in the column was 20-25 mbar. The reboiler used was a 320 W Barnstead Electrothermal EMA stirring heating mantle model. The reboiler was operated on setting 5/10. The heating power was thus 5/10*320 W=160 W. The reflux ratio RD=2 was held constant over the whole distillation period. Schematic set-up of the instrument is shown in FIG. 3.

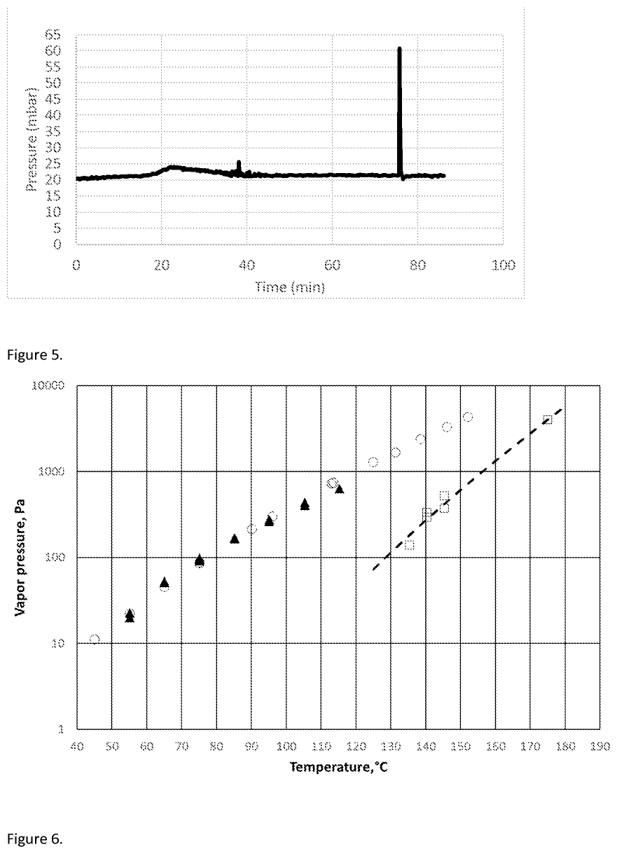

The temperature during the distillation was followed (FIG. 4). These were obtained from two temperature probes, one in the bottom flask (continuous line in FIG. 4), at the base of the distillation column (dotted line in FIG. 4) and the other located at the top of the column. In addition, pressure was followed during the distillation (FIG. 5). The two distinct spikes in pressure are caused by the taking of the distillate fractions (FIG. 5).

The distilled m-% was 91.1 and the lost m-% was 2.8 calculated from the mass of the original sample.

The distillation was started from total reflux after the temperature of the column was observed to be relatively stable. The reflux ratio was then set to 2 for the duration of the distillation and fractions were collected as shown in Table 1.

TABLE 1
Details of the collected distillation fractions. Btm = bottoms fraction.

| | | | Temperature |
|---|---|---|---|
| Sample | Mass of | Pressure | at top of |
| Name | fraction (g) | (mbar) | column (° C.) |
| Dist 2 | 132.27 | 21.3 | 135.6 |
| Dist 3 | 120.81 | 21.4 | 135.9 |
| Dist 4 | 132.5 | 21.1 | 136.9 |
| Dist 5 | 120.11 | 21.2 | 136.2 |
| Dist 6 | 130.73 | 21.1 | 136.5 |
| Dist 7 | 20.25 | 21 | 136.3 |
| Btm | 44.49 | 21 | 135.8 |

The fractions were analysed with NMR to determine the components and composition present. DMSO was used as a solvent and ¹H NMR isotope of chemical used for analysis. The results obtained from the analysis are shown in Table 2.

TABLE 2
Analysis results from NMR

| | | | B, H- | | | A, H- | |
|---|---|---|---|---|---|---|---|
| sample | | | mTBD-2 | mTBD | TBD | mTBD-1 | |
| name | solvent | Info | [mole %] | [mole %] | [mole %] | [mole %] | additional impurities |
| feed | DMSO | no water | Trace¹ | 97.09 | 2.91 | Trace¹ | ¹: small impurities (from |
| | | | | | | | synthesis); amount is <0.5% |
| Dist 1 | | small | 18.30 | 65.36 | 0 | 16.34 | |
| Dist 2 | | amount of | 1.94 | 97.09 | 0 | 0.97 | |
| Dist 3 | | water | 0 | ~100 | 0 | 0 | |
| Dist 4 | | | 0 | ~100 | 0 | 0 | |
| Dist 5 | | | 0 | ~100 | 0 | 0 | |
| Dist 6 | | | 0 | ~100 | 0 | 0 | |
| Dist 7 | | | 0 | ~100 | 0 | 0 | |
| Btm | | | 0 | 48.78 | 51.22 | 0 | Contains other small |
| | | | | | | | amount of impurities |

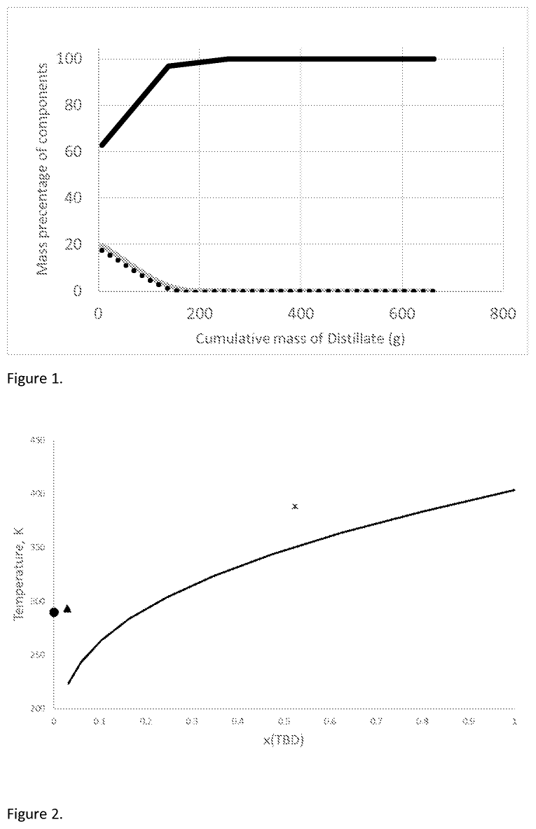

FIG. 1 shows how the distillate compositions changes over time. It can be observed that most of the impurities can be removed at the beginning of the distillation and pure mTBD can be obtained through the distillation process from the third distillation fraction onwards.

The amount of pure mTBD collected could be further improved. The amount for the first distillation fraction that was collected could be increased. It can be observed that the impurities mainly exist at the beginning of the distillation and by increasing the volume of distillate collected initially, there should be less impurities existing in the subsequent fractions. Alternatively, the number of fractions collected could be increased while the volume collected per fraction could be decreased at the beginning of the distillation.

The first two fractions contain a small amount of water and hydrolysis product. At reduced pressure distillation the water cannot be condensed, and it is transferred to the trap with hydrolysis products. The distillate fractions from 3 to 7 were very pure mTBD.

The obtained result was verified by determining the vapor pressure of TBD, which is significantly lower than the vapor pressure of mTBD (FIG. 6).

The sample can be dried if considered necessary before distillation. Water in the sample tends to produce hydrolysis products of mTBD, especially in elevated temperatures. Therefore, removal of excess water may be beneficial for improving the yield of the pure mTBD. In addition, in some cases water needs to be removed from the system before vacuum pumps can be utilized to prevent damages to the pump.

### CITATIONS

### Non-Patent Literature

Zachariah Steven Baird, Artur Dahlberg, Petri Uusi-Kyyny,Nahla Osmanbegovic, Joanna Witos, Jussi Helminen, Daniel Cederkrantz, Paulus Hyväri, Ville Alopaeus, Ilkka Kilpeläinen, Susanne K. Wiedmer, Herbert Sixta, Physical Properties of 7-Methyl-1,5,7-triazabicyclo[4.4.0]dec-5-ene (mTBD) International Journal of Thermophysics, Volume 40, Issue 71 July 2019 Article number 71. DOI 10.1007/s10765-019-2540-2
Sherif Elsayed, Jussi Helminen, Sanna Hellsten, Chamseddine Guizani, Joanna Witos, Marja Rissanen, Antti H. Rantamäki, Paulus Hyväri, Pauliina Varis, Susanne K. Wiedmer, Ilkka Kilpeläinen, and Herbert Sixta. Recycling of Superbase-Based Ionic Liquid Solvents for the Production of Textile-Grade Regenerated Cellulose Fibers in the Lyocell Process, ACS Sustainable Chem. Eng. 2020, 8, 37, 14217-14227 https://doi.org/10.1021/acssuschemeng.0c07773

1. A method for separating mTBD from a sample comprising mTBD and TBD, wherein the method comprises the steps of filtering the sample, and subsequently distilling the sample.

2. The method according to claim 1, wherein the temperature during filtration is 10-30° C.

3. The method according to claim 1, wherein the TBD in the sample is precipitated to obtain a precipitate, and the precipitate is removed prior to distillation.

4. The method according to claim 1, wherein the pressure during distillation is below 101.325 kPa.

5. The method according to claim 1, wherein the pressure during distillation is 1-10 kPa.

6. The method according to claim 1, wherein the distilling is performed in a distillation device selected from the group consisting of batch distillation column, continuous distillation column, short path distillation unit, thin film evaporator or any combination thereof.

7. The method according to claim 1, wherein the sample is dried before distillation.

8. The method according to claim 1, wherein the sample comprises 5 mol-% TBD or less after the filtering, calculated from the combined moles of mTBD and TBD.
